(19) United States
(12) Patent Application Publication
(10) Pub. No.: US 2012/0045493 A1
(43) Pub. Date: Feb. 23, 2012

(54) Solid Cosmetic Composition with Structurant and Electrolyte Solution

(76) Inventors: Christine Popoff, Morganville, NJ (US); Heng Cai, Skillman, NJ (US); Vijay Joshi, Livingston, NJ (US)

(21) Appl. No.: 13/264,835

(22) PCT Filed: May 11, 2010
(86) PCT No.: PCT/US2010/034334

§ 371 Date: Oct. 17, 2011

Related U.S. Application Data
(60) Provisional application No. 61/178,598, filed on May 15, 2009.

(51) Int. Cl. A61K 8/06; A61Q 17/04; A61Q 15/00; A61P 17/10; A61K 9/107; A61K 9/14

(52) U.S. Cl. 424/401; 424/489; 424/499; 424/501

(57) ABSTRACT

A water soluble cosmetic or pharmaceutical or pharmaceutical actives in an electrolyte solution which, when impregnated with one or more retentive fillers (such as, for example, silica or hydrophobically modified silica, lamellar filler, crosspolymer, cellulosic, resins, or silicone elastomers) can form stable solid compositions with structurants. Such solid compositions do not require a higher buffer pH or leave residues upon application to the skin. Solid compositions according to the present invention are suitable for cosmetic and pharmaceutical purposes such as antiperspirants, deodorants, lip products, and other topical applications.

This application claims priority from copending PCT application number PCT/US2010/034334, filed May 11, 2010, and claiming priority from U.S. provisional application No. 61/178,598, filed May 15, 2009, now expired, the entire disclosure of which is hereby incorporated by reference.

Cosmetic compositions, such as those in the form of stick or roll-on, and semi-solid cosmetic compositions, can be either water or oil based. Water based stick cosmetic compositions are often formed using a gellant as a structurant. This often requires use of a buffering agent to maintain a specific pH range and to mitigate degradation of the structurant.

Traditional oil based cosmetic compositions usually require incorporation of anhydrous cosmetic actives, such as powders. Such oil based cosmetic compositions either in a stick suspension or liquid suspension can be chalky and leave noticeable residue upon application to the skin.

Cosmetic compositions may incorporate water soluble cosmetic or pharmaceutical actives by forming water-in-oil or oil-in-water emulsions. Such water-in-oil and oil-in-water emulsions can be wet and sticky after application. In addition, water-in-oil emulsions require the addition of emulsifiers for structure/stability to the formula. These may reduce efficacy and/or release of the cosmetic actives incorporated in the internal phase.

The present invention relates to the surprising discovery that water soluble cosmetic or pharmaceutical or pharmaceutical actives in an electrolyte solution which, when impregnated with one or more retentive fillers (such as, for example, silica or hydrophobically modified silica, lamellar filler, crosspolymer, cellulosic, resins, or silicone elastomers) can form stable solid compositions with structurants. Such solid compositions do not require a higher buffer pH or leave residues upon application to the skin. Solid compositions according to the present invention are suitable for cosmetic and pharmaceutical purposes such as antiperspirants, deodorants, lip products, and other topical applications.

One object of the present invention is to provide stable cosmetic compositions incorporating actives, in an electrolyte solution, that are readily released.

Another object of the invention is to provide stable solid cosmetic compositions that do not require buffering to a lower acidity.

Yet another object of the invention is to provide stable solid cosmetic compositions that do not leave residues upon application to the skin.

In an embodiment a cosmetic or pharmaceutical composition has one or more cosmetic or pharmaceutical actives in a concentration of about 1% to 60% of the total formula wherein said cosmetic or pharmaceutical active is solubilized in an electrolyte solution free of water-absorbing polymers, dipropylene glycol, and emulsifiers and is impregnated within one or more retentive fillers, has one or more structurants having a melting point higher than about 50° C., and an oil phase selected from the group consisting of organic or silicone compounds or a combination of both such that the oil phase is greater than about 10% w/w and less than about 80% w/w, and said composition is capable of forming a solid composition.

The cosmetic or pharmaceutical composition has a retentive filler selected from the group comprising silica, hydrophobically modified silica, lamellar filler, crosspolymer, cellulosic, resins, silicone elastomer and any combination thereof.

The composition has an oil phase comprised of volatile oils, non-volatile oils, or a combination of both.

The composition of claim 1, wherein the composition optionally includes excipients, pigments, essential oils, natural oils, wash-off agents, preservatives, gums, powders, synthetic ingredients, fragrances, masking agents, high refractive index material, sunscreen and combinations thereof.

The composition contains a structurant is selected from the group consisting of waxes, fiber-forming gellants, silicone waxes and combinations thereof.

The composition is prepared by a process comprising:

a. add active and silica powder;

b. optionally mix and add a portion of (a) to the oil phase;

c. add (b) to the structurant and optionally cool;

d. reheat (c) and add the remaining (a);

e. optionally add additional ingredients; and

f. heat (e) until it forms a pourable liquid and pour in molds to obtain a solid composition. The process can be modified so step (c) is merged with step (b) or step (b) is merged with step (a) and step (d) comprises only reheating.

### DETAILED DESCRIPTION

The present invention relates to the surprising discovery that water soluble cosmetic or pharmaceutical active ingredients solubilized in an aqueous electrolyte solution and impregnated within one or more retentive fillers, such as silica or hydrophobically modified silica, lamellar filler, crosspolymer, cellulosic, resins, or silicone elastomers, or any combination thereof can form stable solid topical compositions, with structurants, typically used for gelling oil based, anhydrous compositions. Such solid compositions do not require a higher buffer pH or leave residues upon application to the skin. The solid compositions according to the present invention are suitable for cosmetic and pharmaceutical purposes such as antiperspirants, deodorants, lip products, and topical pharmaceutical applications.

The terms used in this specification generally have their ordinary meanings in the art, within the context of the invention, and in the specific context where each term is used. Certain terms are discussed below, or elsewhere in the specification, to provide additional guidance to the practitioner in describing the compounds, compositions, and methods of the invention and how to make and use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. The use of examples anywhere in this specification, including examples of any terms discussed herein, is illustrative only, and in no way limits the scope and meaning of the invention or of any exemplified term. Likewise, the invention is not limited to the examples presented.

As used herein, the term “about” or “approximately” generally means within 20 percent, preferably within 10 percent, and more preferably within 5 percent of a given value or range.

As used herein, when a composition is essentially free of an ingredient (such as water-absorbing polymers, dipropylene glycol, or emulsifiers), it includes the absence of an ingredient added to a formula or if a fragrance is present in the formula the free ingredient is included as part of the fragrance but not as a separate ingredient.

Soluble Cosmetic and Pharmaceutical Active

The composition of the invention contains one or more water soluble cosmetic or pharmaceutical active ingredients solubilized in an electrolyte solution. The term “electrolyte” refers to a substance which can be dissolved in water or other solvents to form a solution capable of effecting ionic conduction. The term “electrolyte solution” refers to a solution in which at least one kind of such a substance is dissolved. The composition may contain from about 1-60%, by weight of the total composition of water soluble cosmetic or pharmaceutical actives solubilized in the electrolyte solution. The electrolyte solution must be capable of solubilizing, at least in part, the cosmetic or pharmaceutical actives that are used in the composition. The concentration of the soluble actives in the electrolyte solution is generally below or at saturation. However, in certain embodiments, small amounts of soluble actives may not be dissolved, i.e., may remain in the crystalline or suspension form.

Oil Soluble Actives can be Optionally Added to the Oil Phase.

The electrolyte solution should be essentially free of dipropylene glycol (DPG), as well as, sodium polyacrylate, potassium polyacrylate or other surfactant free water soluble polymers.

Actives of the present invention may be in the form of an antiperspirant. Suitable antiperspirant actives are known in the art. The term “antiperspirant active” also known as “antiperspirant salt” refers to any compound or composition having antiperspirant activity. Examples of antiperspirant actives include, but are not limited to, astringent metallic salts such as the inorganic and organic salts of aluminum, zirconium, and zinc, and mixtures thereof (e.g., aluminum halides, aluminum hydroxide halides, zirconyl oxide halides, zirconyl hydroxy halides, and mixtures thereof). Aluminum salts may include those of the formula: Al₂(OH)ₐ(Cl)_{b}.xH₂O wherein a is from about 2 to 5; a+b=6; x is from about 1 to about 6; and wherein a, b, and x may have non-integer values. Zirconium salts include those of the formula: ZrO(OH)₂₋ₐClₐ.xH₂O wherein a is from about 1.5 to about 1.87; x is from about 1 to about 7; and wherein a and n may have non-integer values.

Specific examples of aluminum and zirconium salts include, but are not limited to, aluminum chloride, aluminum chlorohydrate, aluminum chlorohydrex PEG, aluminum chlorohydrex PG, aluminum dichlorohydrate, aluminum dichlorohydrex PEG, aluminum dichlorohydrex PG, aluminum sesquichlorohydrate, aluminum sesquichlorohydrex PEG, aluminum sesquichlorohydrex PG, aluminum zirconium octachlorohdrate, aluminum zirconium octachloroydrex GLY, aluminum zirconium pentachlorohydrate, aluminum zirconium pentachlorohydrex GLY, aluminum zirconium tetrachlorohydrate, aluminum zirconium tetrachlorohydrex GLY, aluminum zirconium trichlorohydrate, aluminum zirconium trichlorohydrex GLY, and mixtures thereof.

Zirconium salts may include those in the form of complexes that also contain aluminum and glycine, in particular, aluminum zirconium tetrachlorohydrex gly. The antiperspirant salts used in the composition of the invention can act as the electrolyte in the solution and additional electrolyte are optional. While, the antiperspirant salts may be completely dissolved in the electrolyte solution, in some cases small amounts of salts may not be dissolved, i.e., may remain in the crystalline form.

Actives also include antiperspirant actives stabilized by Betaine. Betaine is known under IUPAC nomenclature as 1-carboxy-N,N,N-trimethylmethanaminium hydroxide-inner salt. It is also known as carboxymethyl-trimethyl-ammonium betaine, (carboxymethyl) trimethylammonium hydroxide-inner salt, glycine betaine, glycol betaine, glycyl betaine, trimethylglycol, or trimethylglycine. Betaine has the formula C₅H₁₁NO₂:

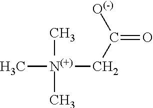

Examples of suitable Betaine stabilized antiperspirant actives include aluminum zirconium tetrasalt or octasalt buffered by Betaine. Betaine stabilized antiperspirant actives may be free of glycine and/or having low metal to chloride ratios. Examples of commercially available glycine free, low metal to chloride ratio tetrasalts and octasalts and include (but are not limited to) Rezal AZP 955 CPG and Rezal AZP 885 respectively from Reheis Chemical Company, Berkeley Heights, N.J.

Actives may also include antiperspirant actives stabilized by a water soluble calcium salt, also known as calcium enhanced aluminum-zirconium chlorohydrate (CEAZCH) salts. Suitable calcium salts include calcium chloride, calcium bromide, calcium nitrate, calcium citrate, calcium formate, calcium acetate, calcium gluconate, calcium ascorbate, calcium lactate, calcium glycinate, calcium carbonate, calcium sulfate, calcium hydroxide, and mixtures thereof.

The composition of the invention may be in the antiperspirant or deodorant form, or it is possible for the composition to contain both a mixture of antiperspirant salts and one or more deodorant ingredients. If the composition is a deodorant or combination antiperspirant or deodorant, the composition may contain one or more deodorant active ingredients. Deodorant actives include antibacterial or antimicrobial agents, as for example without limitation, triclosan, sodium phenolsulfate, phenol, 2-amino-2-methyl-1-propanol (AMP), methylbenzethonium chloride, laurylpyridinium chloride, hexachlorophene, sodium bicarbonate, chloroxylenol, bromochlorophene, cetylpyridinium chloride, benzethonium chloride, ethylhexylglycerin and the like as well as silver halides and various zinc salts (for example, zinc ricinoleate). Actives include natural based deodorizers such as the essential oil of thyme and other fragrance components that inhibit or slow down the growth and multiplication of odor causing bacteria. The deodorant or bacteriostat can, illustratively, be included in the composition in an amount of 0-5%,

Embodiments of the present invention may contain alpha hydroxyl acid as actives. The term “alpha hydroxy acid,” “α-hydroxy acid,” or “AHA” refers to carboxylic acids in which one hydroxyl group is attached to the alpha carbon of the acid. Alpha hydroxy acids have the general formula:

R₁R₂C(OH)COOH

wherein R₁ and R₂ are independently H, F, Cl, Br, alkyl, aralkyl, or aryl group of saturated or unsaturated, isomeric or non-isomeric, straight or branched chain or cyclic form, having 1 to 25 carbon atoms. In addition R₁ and R₂ may each carry one or more OH, CHO, COOH, or alkoxy groups having 1 to 9 carbon atoms. Alpha hydroxy acids may exist as stereoisomers as D, L, and DL forms when R₁ and R₂ are not identical. The alpha hydroxy acids used in the composition of the invention can act as the electrolyte in the solution and additional electrolyte are optional.

Alpha hydroxy acids may be natural or synthetic. Natural alpha hydroxy acids are found in fruits or derived from fruits or milk sugars. Suitable alpha hydroxy acids include but are not limited to allonic acid, altronic acid, arabinoic acid, atrolactic acid, benzilic acid, 4-chloromandelic acid, citric acid, 3,4-dihydroxymandelic acid, erythronic acid, ethyl pyruvate, galactoheptonic acid, galactonic acid, galacturonic acid, glucoheptonic acid, gluconic acid, gluconolactone, glucuronic acid, glucuronolactone, glyceric acid, glycolic acid, gulonic acid, 2-hydroxybutanoic acid, 2-hydroxypentanoic acid, 2-hydroxyhexanoic acid, 2-hydroxyheptanoic acid, 2-hydroxyactanoic acid, 2-hydroxynonanoic acid, 2-hydroxydecanoic acid, 2-hydroxyundecanoic acid, 4-hydroxymandelic acid, 3-hydroxy-4-methoxymandelic acid, 4-hydroxy-3-methoxymandelic acid, α-hydroxyarachidonic acid, α-hydroxybutyric acid, α-hydroxyisobutyric acid, α-hydroxylauric acid, α-hydroxymyristic acid, α-hydroxypalmitic acid, α-hydroxystearic acid, 3-(2′-hydroxyphenyl)lactic acid, 3-(4′-hydroxyphenyl)lactic acid, idonic acid, lactic acid, lyxonic acid, malic acid, mandelic acid, mannoic acid, methyllactic acid, methyl pyruvate, mucic acid, α-phenylactic acid, α-phenylpyruvic acid, pyruvic acid, ribonic acid, saccharic acid, talonic acid, tartaric acid, tartronic acid, threonic acid, xylonic acid, cosmetically or pharmaceutically acceptable salts thereof, derivatives thereof, and mixtures thereof. Suitable alpha hydroxy acids may also include alpha hydroxy and botanical complex, glycomer in crosslinked fatty acids alpha nutrium, sugar cane extract, mixed fruit acid, tri-alpha hydroxy fruit acid, triple fruit acid, derivatives thereof, and mixtures thereof.

Embodiments of the present invention may also contain actives used for acne, such as benzoyl peroxide, retinoids, actives used for psoriasis as well as topical antibacterial agents which include but are not limited to antibiotics. Sunscreen actives such as octylmethoxycinnamate, avobenzone, octylphthalate, zinc oxide and other sunscreen actives listed in the sunscreen monograph can be incorporated in the present embodiment as well.

### The Retentive Filler

Embodiments of the present invention include at least one retentive filler. The term “retentive filler” refers to particulates that have channels, interstices, matrices, or are in a lamellar configuration, and are capable of imbibing the solubilized antiperspirant or deodorant active within its free spaces. Solubilized active ingredients impregnated into the retentive filler may be expressed from it, either in whole or in part, upon application of pressure, such as the pressure that is applied when the composition is applied to the axilla area. A variety of retentive fillers may be present, and ranges may be from about 0.1-95%, in certain embodiments about 0.2-50%, and in other embodiments about 0.5-5% by weight of the total composition. The cosmetic active to retentive filler ratio may be less than about 142:1, and in certain embodiments between about 99:1 and 7:1, by weight.

Exemplary retentive fillers are further described herein. Retentive fillers may have particle sizes ranging from about 0.01-1000 microns, in certain embodiments about 0.1-500 microns, and in other embodiments from about 1-100 microns.

Various types of silicas or silicates may be used as retentive fillers in certain embodiments of the present invention. Such silicates may include those typically found in lamellar or porous form such as silica, fumed silica, calcium silicate, aluminum silicate, hydrated silica, magnesium aluminum silicate, magnesium trisilicate, silica silylate, or silicas that are substituted with hydrophobic or hydrophilic groups such as C₁₋₆ alkyl groups, C₁₋₆ alkoxy groups, and the like. In certain embodiments of the invention, retentive fillers include silica, silica silylate or mixtures thereof.

Lamellar fillers may also be used as retentive fillers. The term “lamellar” with respect to filler, refers to particulates in the form of plates or sheets that may be joined in one or more places. Typically such sheets are layered on top of each other when in the dry state, but are capable of separating when exposed to polar solvents such as water. Examples of fillers that are in the lamellar form include talc, mica, titanated mica, boron nitride, bentonite, diatomaceous earth, fuller's earth, hectorite, kaolin, montmorillonite, attapulgite, or quaternized clays (such as hectorites or bentonites that are reacted with quaternary ammonium compounds, for example, quaternium-18 hectorite, quaternium-18 bentonite, and the like).

A wide variety of crosspolymers are suitable as lamellar fillers for certain embodiments of the present invention, including organic polymers, silicone polymers, or copolymers of organic and silicone monomers which are not water absorbing. The term “crosspolymer” generally refers to polymer containing crosslinked groups. Crosslinking can cause the polymer to form a matrix having inner channels or interstices that are capable of imbibing solubilized active ingredients. Additionally these crosspolymers may provide desirable application aesthetics.

Organic polymers include but are not limited to polymers of polymerized ethylenically unsaturated monomers where at least some of the monomers have crosslinkable groups which crosslink during or soon after polymerization of the polymer. The final polymer may be a homopolymer, copolymer, terpolymer, or graft or block copolymer, and may contain monomeric units such as acrylic acid, methacrylic acid or their simple C₁₋₃₀ alkyl esters, styrene, ethylenically unsaturated monomer units such as ethylene, propylene, butylene, etc., vinyl monomers such as vinyl chloride, styrene, and so on.

In some cases, the crosspolymer may contain one or more monomers which are esters of acrylic acid or methacrylic acid, including aliphatic esters of methacrylic acid like those obtained with the esterification of methacrylic acid or acrylic acid with an aliphatic alcohol of 1 to 30, preferably 1 to 20, more preferably 1 to 8 carbon atoms. If desired, the aliphatic alcohol may have one or more hydroxy, carboxy, or carboxylic acid groups. Also suitable are methacrylic acid or acrylic acid esters esterified with moieties containing alicyclic or bicyclic rings such as cyclohexyl or isobornyl, for example.

The ethylenically unsaturated monomer may be mono-, di-, tri-, or polyfunctional as regards the addition-polymerizable ethylenic bonds. A variety of ethylenically unsaturated monomers are suitable.

Examples of suitable monofunctional ethylenically unsaturated monomers include, but are not limited to, those of the formula (I):

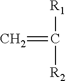

wherein R₁ is H, OH, a C₁₋₃₀ straight or branched chain alkyl, aryl, aralkyl; R₂ is a pyrrolidone, a C₁₋₃₀ straight or branched chain alkyl, or a substituted or unsubstituted aromatic, alicyclic, or bicyclic ring where the substitutents are C₁₋₃₀ straight or branched chain alkyl, or COOM or OCOM wherein M is H, OH, a C₁₋₃₀ straight or branched chain alkyl, pyrrolidone, or a substituted or unsubstituted aromatic, alicylic, or bicyclic ring where the substitutents are C₁₋₃₀ straight or branched chain alkyl which may be substituted with one or more hydroxyl, carboxy, carboxylic acid, or other types of groups, or [(CH₂)ₘO]ₙH wherein m is 1-20, and n is 1-200.

Di-, tri- and polyfunctional monomers, as well as oligomers, of the above monofunctional monomers may also be used to form crosspolymers. Suitable difunctional monomers include those having the general formula (II):

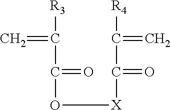

wherein R₃ and R₄ are each independently H, a C₁₋₃₀ straight or branched chain alkyl, aryl, or aralkyl; and X is [(CH₂)ₓO_{y}]_{z} wherein x is 1-20, and y is 1-20, and z is 1-100. Particularly preferred are difunctional acrylates and methacrylates, such as the compound of formula II above wherein R₃ and R₄ are CH₃ and X is [(CH₂)ₓO_{y}], wherein x is 1-4; and y is 1-6; and z is 1-10.

Trifunctional and polyfunctional monomers are also suitable for use in the polymerizable monomers to form polymers used in certain embodiments of the present invention. Examples of such monomers include acrylates and methacrylates such as trimethylolpropane trimethacrylate or trimethylolpropane triacrylate,

Also suitable are polymers formed from the monomer of Formula I, above, which are cyclized, in particular, cycloalkylacrylate polymers or copolymers having the following general formulas:

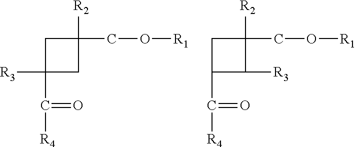

wherein R₁, R₂, R₃, and R₄ are as defined as above. Typically such polymers are referred to as cycloalkylacrylate polymers.

The monomers mentioned herein can be polymerized with various types of organic groups such as propylene glycol, isocyanates, amides, etc.

One type of organic group that can be polymerized with the above monomers includes a urethane monomer. Urethanes are commonly formed by the reaction of polyhydroxy compounds with diisocyanates, as follows:

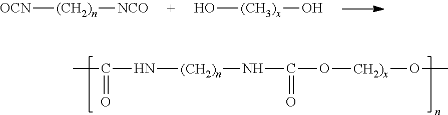

wherein x is 1-1000.

Another type of monomer that may be polymerized with the above comprise amide groups, preferably having the following formula:

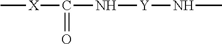

wherein X and Y are each independently linear or branched alkylene having 1 to 40 carbon atoms, which may be substituted with one or more amide, hydrogen, alkyl, aryl, or halogen substituents.

Another type of organic monomer may be alpha or beta pinenes, or terpenes, abietic acid, and the like.

Suitable crosslinked retentive fillers may also be made by polymerizing ethylenically unsaturated monomers which comprise vinyl ester groups either alone or in combination with other monomers including silicon monomers, other ethylenically unsaturated monomers, or organic groups such as amides, urethanes, glycols, and the like. The various types of monomers or moieties may be incorporated into the film-forming polymer by way of free radical polymerization, addition polymerization, or by formation of grafts and blocks which are attached to the growing polymer chain according to processes known in the art. Preferably the film forming polymer is an organic synthetic polymer obtained by polymerizing ethylenically unsaturated monomers comprised of vinyl ester groups and optionally organic or silicon groups or other types of ethylenically unsaturated monomers.

Other types of retentive fillers may be polymerized and crosslinked polymers having one or more vinyl ester monomers having the following general formula:

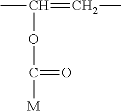

wherein M is H, or a straight or branched chain C₁₋₁₀₀ alkyl, preferably a C₁₋₅₀ alkyl, more preferably a C₁₋₄₅ alkyl which may be saturated or unsaturated, or substituted or unsubstituted, where the substituents include hydroxyl, ethoxy, amide or amine, halogen, alkyloxy, alkyloxycarbonyl, and the like. Preferably, M is H or a straight or branched chain alkyl having from 1 to 30 carbon atoms. The retentive filler may be a homopolymer or copolymer having the vinyl ester monomers either alone or in combination with other ethylenically unsaturated monomers, organic groups, or silicon monomers.

Suitable other monomers that may be copolymerized with the vinyl ester monomer include those having siloxane groups, including but not limited to those of the formula:

SiOₙ

wherein n ranges from 1-1,000,000. The silicon monomers are preferably polymerized into a siloxane polymer then attached to the polymer chain by attaching a terminal organic group having olefinic unsaturation such as ethylene or propylene, to the siloxane, then reacting the unsaturated group with a suitable reactive site on the polymer to graft the siloxane chain to the polymer.

Various types of organic groups may be polymerized with the vinyl ester monomers including but not limited to urethane, amide, polyalkylene glycols, and the like as set forth above.

The vinyl ester monomers may also be copolymerized with other ethylenically unsaturated monomers that are not vinyl esters, including those set forth above.

In certain embodiments it is preferred that the crosspolymer is a polymer of crosslinked methacrylic acid esters or crosslinked polystyrene. One type of crosslinked methacrylic acid ester is a crosslinked polymethylmethacrylate having the INCI name methyl methacrylate crosspolymer, which may be purchased from Presperse Inc., in Piscataway, N.J., and is available under the tradename Ganzpearl. Other types of crosspolymers that are suitable include allyl methacrylates crosspolymer or HDI trimethylol, hexyllactone crosspolymer, the latter being a polymer that is the cross-linked condensation polymer formed from the reaction of hexyldiisocyanate with the esterification product of trimethyloipropane with 6 to 7 moles of hexyllactone. One commercial source of HDI trimethylollactone crosspolymer is Kobo Products Inc., sold under the trade name BPD-800, which is a particulate material. One commercial source for allyl methacrylates crosspolymer is Amcol Health & Beauty Solutions under the trade name Poly Pore E 200. One commercial source for silicone crosspolymer such as cyclomethicone and dimethicone crosspolymer is Dow Corning Corporation under the trade names DC 9040 and DC 9042.

Cellulosics may also be suitable retentive fillers. Such cellulosics are polymers containing repeating cellulose units, such as starches or modified starches, either as homopolymers or copolymerized with other cellulose monomers or organic monomers. Such cellulosics may also contain alkali metal or alkaline earth metal substituents. The cellulosics may be substituted with one or more groups that confer hydrophobicity or hydrophilicity. Examples of suitable cellulosics include starch, starch substituted with C₁₋₁₀ alkyl or alkoxy groups including methyl, ethyl, propyl, methoxy, ethoxy, propoxy, etc., or starch substituted with alkali or alkaline earth metals such as sodium, potassium, magnesium, aluminum, and so on.

Also suitable are cellulosics such as starch that may be copolymerized with succinimates, succinates, or succinimides, or derivatives thereof, including materials such as aluminum starch octenylsuccinate, and the like. Particularly preferred starches are hydroxypropyl starch, hydroxyethyl starch, sodium carboxymethyl starch, aluminum starch octenylsuccinate, corn starch, rice starch, microcrystalline cellulose, maltodextrin, aluminum starch, dextran, glyceryl starch, and the like.

Also suitable as retentive fillers are various resins including silicone resins, organic resins, or copolymers thereof, so long as the resin is capable of imbibing solubilized active ingredients.

Typically silicone resins are at least partially crosslinked and include those referred to as T or Q resins. The term “T” generally means “trifunctional siloxy unit” and in standard silicone nomenclature a “T” unit has the general formula:

R₁SiO_{3/2}

wherein R₁ is C₁₋₃₀, preferably C₁₋₁₀, more preferably, C₁₋₄ straight or branched chain alkyl, which may be substituted with phenyl or one or more hydroxyl groups; phenyl; alkoxy (preferably C₁₋₂₂, more preferably C₁₋₆ alkyl); or hydrogen. The SiO_{3/2} designation means that the silicon atom is bonded to three oxygen atoms when the unit is copolymerized with one or more of the other units. For example when R₁ is methyl the resulting trifunctional unit is of the formula:

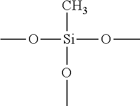

When this trifunctional unit is polymerized with one or more of the other units, the silicon atom shares three oxygen atoms with other silicon atoms, i.e. will share three halves of an oxygen atom.

The term “tetrafunctional siloxy unit” is generally designated by the letter “Q” in standard silicone nomenclature. A “Q” unit has the general formula:

SiO_{4/2}

The SiO_{4/2} designation means that the silicon shares four oxygen atoms (i.e. four halves) with other silicon atoms when the tetrafunctional unit is polymerized with one or more of the other units. The SiO_{4/2} unit is best depicted as follows:

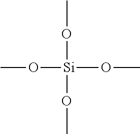

The resin may contain only T or Q units, or may be copolymerized with other siloxane units such as M or D units.

The term “monofunctional unit” or “M” means a siloxy unit that contains one silicon atom bonded to one oxygen atom, with the remaining three substituents on the silicon atom being other than oxygen. In particular, in a monofunctional siloxy unit, the oxygen atom present is shared by 2 silicon atoms when the monofunctional unit is polymerized with one or more of the other units. In silicone nomenclature used by those skilled in the art, a monofunctional siloxy unit is designated by the letter “M”, and means a unit having the general formula:

R₁R₂R₃SiO_{1/2}

wherein R₁, R₂, and R₃ are each independently C₁₋₃₀, preferably C₁₋₁₀, more preferably C₁₋₄ straight or branched chain alkyl, which may be substituted with phenyl or one or more hydroxyl groups; phenyl; alkoxy (preferably C₁₋₂₂, more preferably C₁₋₆ alkyl; or hydrogen. The SiO_{1/2} designation means that the oxygen atom in the monofunctional unit is bonded to, or shared, with another silicon atom when the monofunctional unit is polymerized with one or more of the other types of units. For example, when R₁, R₂, and R₃3 are methyl the resulting monofunctional unit is of the formula:

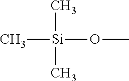

When this monofunctional unit is polymerized with one or more of the other units the oxygen atom will be shared by another silicon atom, i.e. the silicon atom in the monofunctional unit is bonded to ½ of this oxygen atom.

The term “difunctional siloxy unit” is generally designated by the letter “D” in standard silicone nomenclature. If the D unit is substituted with substituents other than methyl the “D” designation is sometimes used, which indicates a substituent other than methyl. For purposes of this disclosure, a “D” unit has the general formula:

R₁R₂SiO_{2/2}

wherein R₁ and R₂ are defined as above. The SiO_{1/2} designation means that the silicon atom in the difunctional unit is bonded to two oxygen atoms when the unit is polymerized with one or more of the other units. For example, when R₁ and R₂ are methyl the resulting difunctional unit is of the formula:

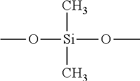

When this difunctional unit is polymerized with one or more of the other units the silicon atom will be bonded to two oxygen atoms, i.e. will share two one-halves of an oxygen atom.

The siloxane resins that form suitable retentive fillers generally comprise a majority of T or Q units, either alone or in combination with minor amounts of M or D units. The term “major amount” meaning that the T or Q units in the polymer are present such that the resulting polymer has sufficient porosity. The term “minor amount” means that the M or D units, if present, are not present in an amount that provides a particulate that is does not have the required degree of porosity. T or MT silicones are often referred to as silsesquioxanes, and, in the case where M units are present, methylsilsesquioxanes. One type of T silicone that may be suitable for use as the retentive filler has units of the following general formula:

(R₁SiO_{3/2})ₓ

where x ranges from about 1 to 100,000, preferably about 1-50,000, more preferably about 1-10,000, and wherein R₁ is as defined above. Such MT silicones are generally referred to as polymethylsilsesquioxane which are silsesquioxanes containing methyl groups.

Examples of specific polysilsesquioxanes that may be used are manufactured by Wacker Chemie under the Resin MK designation. This polysilsesquioxane is a polymer comprised of T units and, optionally one or more D (preferably dimethylsiloxy) units. This particular polymer may have ends capped with ethoxy groups, and/or hydroxyl groups. Other suitable polysilsesquioxanes that may be used as retentive fillers include those manufactured by Shin-Etsu Silicones and include the “KR” series, e.g. KR-220L, 242A, and so on. These particular silicone resins may contain endcap units that are hydroxyl or alkoxy groups.

Also suitable as retentive fillers are MQ resins, which are siloxy silicate polymers having the following general formula:

[(R₁R₂R₃)₃SiO_{1/2}]ₓ[SiO₂]_{y}

wherein R₁, R₂, and R₃ are each independently a C₁₋₁₀ straight or branched chain alkyl or phenyl, and x and y are such that the ratio of (R₁R₂R₃)₃SiO_{1/2} units to SiO₂ units ranges from about 0.5 to 1 to 1.5 to 1. Preferably R₁, R₂, and R₃ are a C₁₋₆ alkyl, and more preferably are methyl and x and y are such that the ratio of (R₁R₂R₃)₃SiO_{1/2} units to SiO₂ units is about 0.75 to 1. More specifically, the trimethylsiloxysilicate thus formed contains from about 2.4 to 2.9 weight percent hydroxyl groups which is formed by the reaction of the sodium salt of silicic acid, chlorotrimethylsilane, and isopropyl alcohol. The manufacture of trimethylsiloxysilicate is set forth in U.S. Pat. Nos. 2,676,182; 3,541,205; and 3,836,437, the disclosures of which are hereby incorporated by reference. Trimethylsiloxysilicate is available from GE Silicones under the trade name SR-1000, which is a solid, particulate material. Also suitable is Dow Corning 749 which is a mixture of volatile cyclic silicone and trimethylsiloxysilicate.

The siloxane polymeric resins that may be used as retentive fillers in certain embodiments of the present invention may be made according to processes well known in the art. In general siloxane polymers are obtained by hydrolysis of silane monomers, preferably chlorosilanes. The chlorosilanes may be hydrolyzed to silanols and then condensed to form siloxanes. For example, Q units are often made by hydrolyzing tetrachlorosilanes in aqueous or aqueous/alcoholic media to form the following:

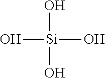

The above hydroxy substituted silane is then condensed or polymerized with other types of silanol substituted units such as:

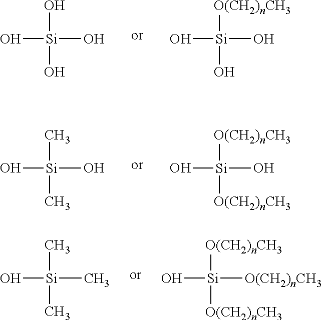

wherein n is 0-10, preferably 0-4. Because the hydrolysis and condensation may take place in aqueous or aqueous/alcoholic media wherein the alcohols are preferably lower alkanols such as ethanol, propanol, or isopropanol, the units may have residual hydroxyl or alkoxy functionality as depicted above. Preferably, the resins are made by hydrolysis and condensation in aqueous/alcoholic media, which provides resins that have residual silanol and alkoxy functionality. In the case where the alcohol is ethanol, the result is a resin that has residual hydroxy or ethoxy functionality on the siloxane polymer. Silicone polymers that may be used in accordance with the present invention are generally made using the methods set forth in Silicon Compounds (Silicones), Bruce B. Hardman, Arnold Torkelson, General Electric Company, Kirk-Othmer Encyclopedia of Chemical Technology, Volume 20, Third Edition, pages 922-962, 1982, which is hereby incorporated by reference in its entirety. Silicone elastomers may be suitable retentive fillers or aesthetic modifier. Silicone elastomers are generally cross-linked organosiloxane compounds prepared by reacting a dimethyl methylhydrogen siloxane with a crosslinking group comprised of a siloxane having an alkylene group having terminal olefinic unsaturation or with an organic group having an alpha or omega diene. Examples of suitable silicone elastomers include but are not limited to Dow Corning 9040, sold by Dow Corning, and various elastomeric silicones sold by Shin-Etsu under the KSG tradenames including KSG 15, KSG 16, KSG 19 and so on. Silicone crosspolymers (elastomers) can be comprised of cyclomethicone (and) dimethicone crosspolymer made with an .ident.Si—H containing polysiloxane and an alpha, omega-diene of formula CH.sub.2.dbd.CH(CH.sub.2).sub.xCH.dbd.CH.sub.2, where x=1 20, to form a matrix gel by crosslinking and addition of .ident.Si—H across double bonds in the alpha, omega diene (an example of such a crosspolymer composition being DC-9040 from Dow Corning Corporation (Midland, Mich.) with other types of such crosspolymers (also called elastomers) being described in U.S. Pat. No. 5,654,362, incorporated by reference herein. Examples of additional non-emulsifying elastomers are also given in PCT cases WO 97144010, WO 98/00097, WO 98/00104 and 98/00105. U.S. Pat. No. 5,922,308, herein incorporated by reference. Silicone polymers that may be used in accordance with the present invention are generally made using the methods set forth in Silicon Compounds (Silicones), Bruce B. Hardman, Arnold Torkelson, General Electric Company, Kirk-Othmer Encyclopedia of Chemical Technology, Volume 20, Third Edition, pages 922-962, 1982, which methods are hereby incorporated by reference,

Embodiments of the present invention contain one or more structurants, such as waxes or fiber-forming gellants. Structurants may range from about 0.1-50% and in certain embodiments from about 5-23%, by weight of the total composition. A variety of natural or synthetic waxes may be used as structurants in embodiments of the present invention including animal, vegetable, mineral, or silicone waxes. Generally such waxes have a melting point ranging from about 50 to 125° C., preferably about 50 to 100° C. Examples of animal, vegetable, or mineral waxes include but are not limited to acacia, beeswax, ceresin, cetyl esters, flower wax, citrus wax, carnauba wax, jojoba wax, soybean wax japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, candelilla, grape wax, stearyl alcohol, hydrogenated castor oil, hydrogenated vegetable oil wax, and polyalkylene glycol derivatives thereof such as PEG6-20 beeswax, or PEG-12 carnauba wax. In certain embodiments the wax is stearyl alcohol, hydrogenated castor oil, or mixtures thereof.

Waxes used as structurants also include various types of ethylene homo- or copolymeric waxes such as polyethylene (also referred to as synthetic wax), polypropylene, and mixtures thereof. Examples include but are not limited to polyethylenes sold under the PERFORMALENE™ product line (New Phase Technology, Piscataway, N.J.); MARCUS polyethylenes (for example M200, M300, M500 and M4040) (Marcus Oil and Chemical, Houston, Tex.); HPWax polyethylene waxes (for example, HP CWP 200, HP CWP 500 and HP 400M) (Hase Petroleum Wax Co., Arlington Heights, Ill.). Mixtures of neutral polyethylene wax/polypropylene wax may also be used such as Polarwachs® PT30, Polarwachs® PT70, and Polycerit® AT-grades (TH. C. TROMM GmbH, Germany). Additionally polyethylenes may also be used which are made by the procedures and information found in the art such as British Patent 1 450 285.

Other structurants that can be used for additional hardening of the sticks, include long chain alcohols, for example including but not limited to the Performacol series (available from New Phase Technology, Piscataway, N.J.) 350, 425 and 550 (average carbon chain length ranging from circa 20 to 40 carbons) as well as alcohol ethoxylates such as the Performathox series 420 and 450 that vary but are not limited to a 20% to 50% by weight ethoxylation (also available from New Phase Technology, Piscataway, N.J.)

Structurants include various types of silicone waxes, referred to as alkyl silicones, which are polymers that comprise repeating dimethylsiloxy units in combination with one or more methyl-long chain (C₁₆₋₃₀) alkyl units where the long chain alkyl is preferably a fatty chain that provides a wax-like characteristic to the silicone. Such silicones include, but are not limited to stearoxydimethicone, behenoxy dimethicone, stearyl dimethicone, cetearyl dimethicone, cetyl dimethicone, and so on. Suitable waxes are set forth in U.S. Pat. No. 5,725,845, which is hereby incorporated by reference in its entirety.

Structurants may also be a fiber-forming gellant, such as N-acyl amino acid amides and esters, 12-hydroxystearic acid (12-HSA), lanosterol, or combinations of a sterol and a sterol ester, such as β-sitosterol and γ-oryzanol, a polyesterified cellobiose, especially with a C₈ to C₁₀ aliphatic acid, threitol esters or and selected secondary amides of di- or tri-basic carboxylic acids, (e.g. 2-dodecyl-N,N′-dibutylsuccinimide) by themselves or in combination. One commercial source for N-acyl amino acid amides and esters is Ajinomoto Co., Inc. under the tradenames GB-1 (N-Lauroyl-L-glutamic acid di-n-butylamide, CTFA INCI Name Dibutyl Lauroyl Glutamide) and EB-21 (N-2-Ethylhexanoyl-L-Glutamic acid Dibutylamide, CTFA INCI Name Dibutyl Ethylhexanoyl Glutamide).

Oils

Embodiments of the present invention contain an oil phase. The oil phase is present in the composition in an amount greater than about 10% w/w and less than about 80% w/w. Suitable oils include organic or silicone compounds. The term “oil” when used herein means an ingredient that is pourable at room temperature. Such oils may be volatile or non-volatile. The term “volatile” when used herein means an oil that has a vapor pressure of greater than about 2 mm of mercury at 20° C. The term “non-volatile” means an oil that has a vapor pressure of less than about 2 mm of mercury at 20° C.

A. Volatile Oils

Volatile oils include volatile silicones, paraffinic hydrocarbons, and the like.

(a). Volatile Silicones

Examples of suitable volatile silicones cyclic silicones are of the general formula:

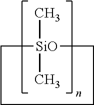

where n=3-6.

Linear and cyclic volatile silicones are also suitable for use in certain embodiments of the present invention. Such silicones have the general formula:

(CH₃)₃Si—O—[Si(CH₃)₂—O]ₙ—Si(CH₃)₃

where n=0, 1, 2, 3, 4, 5, 6, or 7, preferably 0, 1, 2, 3, or 4. Such silicones may include hexamethyldisiloxane, octamethyltrisiloxane, decamethyltetrasiloxane, dodecamethylpentasiloxane, and the like.

Such linear and cyclic volatile silicones are available from various commercial sources including Dow Corning Corporation and General Electric. The Dow Corning volatile silicones are sold under the tradenames DC 244, 245, 246 344, 200, and DC-2-1184 fluids, and may have viscosities ranging from about 0.5 to about 2.0 centistokes (cst) at 25° C. For example, hexamethyldisiloxane primarily comprises silicone having a viscosity of about 0.5 to 0.65 cst, while octamethyltrisiloxane primarily comprises a siloxane having a viscosity of about 1.0 cst, and decamethyltetrasiloxane comprises primarily a siloxane having a viscosity of 1.5 cst, all at 25° C.

(b). Paraffinic Hydrocarbons

Volatile paraffinic hydrocarbons that may be used in the compositions of the invention include various straight or branched chain paraffinic hydrocarbons having 5 to 40 carbon atoms, more preferably 8-20 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, the disclosures of which are hereby incorporated by reference. Preferred volatile paraffinic hydrocarbons have a molecular weight of 70-225, preferably 160 to 190 and a boiling point range of 30 to 320, preferably 60-260° C., and a viscosity of less than 10 centipoise at 25° C., Such paraffinic hydrocarbons are available from EXXON under the ISOPARS trademark, and from the Permethyl Corporation. Suitable C₁₂ isoparaffins are manufactured by Permethyl Corporation under the trade name Permethyl 99A. Another C₁₂ isoparaffin (isododecane) is distributed by Presperse under the trade name Permethyl 99A. Various C₁₆ isoparaffins commercially available, such as isohexadecane (having the trade name Permethyl R), are also suitable. Examples of suitable volatile paraffinic hydrocarbons include isohexadecane, isododecane, or mixtures thereof.

B. Non-Volatile Oils

Examples of suitable non-volatile oils that may be used in accordance with the present invention include organic oils or silicones. Examples include, but are not limited to, esters, hydrocarbon oils, lanolin oil, glyceryl esters of fatty acids, and fluorinated oils. Additional examples of such oils include those disclosed in Cosmetics, Science and Technology 27-104 (Balsam and Sagarin ed. 1972); and U.S. Pat. Nos. 4,202,879 and 5,069,897, the disclosures of which are hereby incorporated by references.

(a). Esters

Organic mono-, di-, or triesters include but are not limited to those set forth below.

(i). Monoesters

Monoesters are defined as esters formed by the reaction of a monocarboxylic acid having the formula R—COOH, wherein R is a straight or branched chain saturated or unsaturated alkyl having 2 to 150 carbon atoms, or phenyl, and an alcohol having the formula R—OH, wherein R is a straight or branched chain saturated or unsaturated alkyl having 2-30 carbon atoms, or phenyl. Both the alcohol and the acid may be substituted with one or more hydroxyl groups. Either one or both of the acid or alcohol may be a “fatty” acid or alcohol, i.e. may have from about 6 to 30 carbon atoms. Examples suitable of monoester oils include hexyldecyl benzoate, hexyl laurate, hexadecyl isostearate, hexydecyl laurate, hexyldecyl octanoate, hexyldecyl oleate, hexyldecyl palmitate, hexyldecyl stearate, hexyldodecyl salicylate, hexyl isostearate, butyl acetate, butyl isostearate, butyl oleate, butyl octyl oleate, cetyl palmitate, ceyl octanoate, cetyl laurate, cetyl lactate, ethylhexyl isononanoate, isostearyl isononanoate, cetyl isononanoate, cetyl stearate, stearyl lactate, stearyl octanoate, stearyl heptanoate, stearyl stearate, and so on. It is understood that in the above nomenclature, the first term indicates the alcohol and the second term indicates the acid in the reaction, e.g., stearyl octanoate is the reaction product of stearyl alcohol and octanoic acid. One commercial source for ethylhexyl isononanoate is Alzo International Inc. under the trade name Dermol 89.

(ii). Diesters

Suitable diesters may be formed from the reaction of a dicarboxylic acid and an aliphatic or aromatic alcohol, or an aliphatic or aromatic alcohol having at least two hydroxyl groups with mono- or dicarboxylic acids. The carboxylic acids may contain from 2 to 150 carbon atoms, and may be in the straight or branched chain, saturated or unsaturated form. The carboxylic acids may be substituted with one or more hydroxyl groups. The aliphatic or aromatic alcohol may also contain 2 to 30 carbon atoms, and may be in the straight or branched chain, saturated, or unsaturated form. Aliphatic or aromatic alcohols may be substituted with one or more substituents such as hydroxyl. Preferably, one or more of the acid or alcohol is a fatty acid or alcohol, i.e. contains 14-22 carbon atoms. The carboxylic acids may also be an alpha hydroxy acid. Examples of suitable diester oils include diisostearyl malate, neopentyl glycol dioctanoate, dibutyl sebacate, di-C₁₂₋₁₃ alkyl malate, dicetearyl dimer dilinoleate, dicetyl adipate, diisocetyl adipate, diisononyl adipate, diisostearyl dimer dilinoleate, disostearyl fumarate, diisostearyl malate, and so on.

(iii). Triesters

Suitable triesters comprise the reaction product of a tricarboxylic acid and an aliphatic or aromatic alcohol, or the reaction product of an aliphatic or aromatic alcohol having three or more hydroxyl groups with various mono-, di-, or tricarboxylic acids. As with the mono- and diesters mentioned above, the acid and alcohol may contain 2 to 150 carbon atoms, and may be saturated or unsatured, straight or branched chain, and may be substituted with one or more hydroxyl groups. Preferably, one or more of the acid or alcohol is a fatty acid or alcohol containing 14 to 22 carbon atoms. Examples of triesters include triarachidin, tributyl citrate, triisostearyl citrate, tri C₁₂₋₁₃ alkyl citrate, tricaprylin, tricaprylyl citrate, tridecyl behenate, trioctyldodecyl citrate, trioctydodecyl citrate dilinoleate, tridecyl behenate, tridecyl cocoate, tridecyl isononanoate, and so on.

(b). Hydrocarbon Oils

Also suitable are one or more hydrocarbons such as paraffins and olefins, preferably those having greater than 20 carbon atoms. Examples of such hydrocarbon oils include C₂₄₋₂₈ olefins, C₃₀₋₄₅ olefins, C₂₀₋₄₀ paraffins, hydrogenated polyisobutene, polyisobutene, mineral oil, pentahydrosqualene, squalene, squalane, and mixtures thereof. Structures of hydrocarbons can vary widely and include but are not limited to aliphatic, acyclic, and aromatic compounds

(c). Lanolin Oil

Lanolin oil or derivatives thereof containing hydroxyl, alkyl, or acetyl groups, such as hydroxylated lanolin, isobutylated lanolin oil, acetylated lanolin, acetylated lanolin alcohol, and so on, may also be used in certain embodiments of the invention.

(d). Glyceryl Esters of Fatty Acids

Embodiments of the invention may comprise naturally occurring glyceryl esters of fatty acids, or triglycerides. Both vegetable and animal sources may be used. Specific examples of such oils include but are not limited to castor oil, lanolin oil, C₁₀₋₁₈ triglycerides, caprylic/capric/triglycerides, coconut oil, corn oil, cottonseed oil, jojoba oil, linseed oil, macadamia nut oil, mink oil, olive oil, palm oil, illipe butter, rapeseed oil, soybean oil, sunflower seed oil, walnut oil, and the like.

Also suitable are synthetic or semi-synthetic glyceryl esters, e.g. fatty acid mono-, di-, and triglycerides which are natural fats or oils that have been modified, for example, acetylated castor oil, or mono-, di- or triesters of polyols such as glyceryl stearate, diglyceryl diiosostearate, polyglyceryl-4 isostearate, polyglyceryl-6 ricinoleate, glyceryl dioleate, glyceryl diisotearate, glyceryl trioctanoate, diglyceryl distearate, glyceryl linoleate, glyceryl myristate, glyceryl isostearate, PEG castor oils, PEG glyceryl oleates, PEG glyceryl stearates, PEG glyceryl tallowates, and so on.

(e). Fluorinated Oils

Also suitable as the oil are various fluorinated oils such as fluorinated silicones, fluorinated esters, or perfluoropolyethers. Particularly suitable are fluorosilicones such as trimethylsilyl endcapped fluorosilicone oil, polytrifluoropropylmethylsiloxanes, and similar silicones such as those disclosed in U.S. Pat. No. 5,118,496, which is hereby incorporated by reference. Perfluoropolyethers like those disclosed in U.S. Pat. Nos. 5,183,589, 4,803,067, 5,183,588, all of which are hereby incorporated by reference, are commercially available from Montefluos under the trademark Fomblin.

Fluoroguerbet esters are also suitable oils. The term “guerbet ester” means an ester which is formed by the reaction of a guerbet alcohol having the general formula:

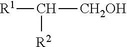

and a fluoroalcohol having the following general formula:

CF₃—(CF₂)ₙ—CH₂—CH₂—OH

wherein n is from 3 to 40, with a carboxylic acid having the general formula:

R³COOH or HOOC—R³—COOH

wherein R¹, R², and R³ are each independently a straight or branched chain alkyl.

Another type of guerbet ester is a fluoro-guerbet ester, which is formed by the reaction of a guerbet alcohol and carboxylic acid (as defined above), and a fluoroalcohol having the following general formula:

CF₃—(CF₂)ₙ—CH₂—CH₂—OH

wherein n is from 3 to 40.

Examples of suitable fluoro guerbet esters are set forth in U.S. Pat. No. 5,488,121, which is hereby incorporated by reference. Suitable fluoro-guerbet esters are also set forth in U.S. Pat. No. 5,312,968, which is also hereby incorporated by reference. One type of such an ester is fluorooctyldodecyl meadowfoamate, sold under the tradename Silube GME-F by Siliech, Norcross Ga.

(f). Silicones

Nonvolatile silicone oils, both water soluble and water insoluble, may also be used in certain embodiments of the invention. Such silicones may have a viscosity ranging from about 10 to 600,000 centistokes, preferably 20 to 100,000 centistokes at 25° C. Suitable water insoluble silicones include amine functional silicones such as amodimethicone; phenyl substituted silicones such as bisphenylhexamethicone, phenyl trimethicone, phenyl dimethicone, or polyphenylmethylsiloxane; dimethicone, alkyl substituted dimethicones such as caprylyl methicone, dimethiconols such as DC 1501 and mixtures thereof.

Such silicones include those having the following general formula:

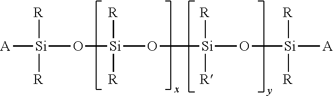

wherein R and R′ are each independently C₁₋₃₀ alkyl, phenyl or aryl, trialkylsiloxy, and x and y are each independently 0-1,000,000 with the proviso that there is at least one of either x or y, and A is siloxy endcap unit or hydroxyl. Preferred is where A is a methyl siloxy endcap unit, in particular trimethylsiloxy, and R and R′ are each independently a C₁₋₃₀ straight or branched chain alkyl, phenyl, or trimethylsiloxy, more preferably a C₁₋₂₂ alkyl, phenyl, or trimethylsiloxy, most preferably methyl, phenyl, or trimethylsiloxy, and resulting silicone is dimethicone, phenyl dimethicone, or phenyl trimethicone. Other examples include alkyl dimethicones such as cetyl dimethicone, and the like wherein at least one R is a fatty alkyl (C₁₂, C₁₄, C₁₆, C₁₈, or C₂₂), and the other R is methyl, and A is a trimethylsiloxy endcap unit.

Other Components

The cosmetic or pharmaceutical compositions may contain excipients which may be used, for example, to modify various properties of the composition and to improve aesthetics. Examples of excipients include wash-off agents, colorants, waxes, oils, preservatives, thickeners, and/or conditioning agents or treatment agents. Commonly used natural and synthetic excipients are described, for example, in International Cosmetic Ingredient Dictionary and Handbook, Twelfth Edition 2008, ISBN-10: 1882621433, (hereinafter “Cosmetic Handbook”) and CTFA ingredient information (http://www.ctfa-online.org/pls/ctfa_online.home), the content of which is hereby incorporated by reference in its entirety.

Wash-off agents are typically used to improve the ease with which the ingredients, particularly the structurants and the non-polar, non-volatile oils, may be washed off. The wash-off agent is preferably in the solid composition in an amount from about 0.1% to about 10%; preferably, from about 0.2% to about 5%; and more preferably, from about 0.5% to about 3%. Suitable wash-off agents include polyoxyethylene ethers having the formula R₁(OCH₂CH₂)ₙOH; polyoxyethylene esters having the formula R₁CO(OCH₂CH₂)ₙOH; polyoxyethylene diesters having the formula R₁CO(OCH₂CH₂)ₙOOCR₂; polyoxyethylene glyceryl esters having the formula (R₁COO)CH₂CH(OH)CH₂(OCH₂CH₂)ₙOH or having the formula HOCH₂CH(OOCR₁)CH₂(OCH₂CH₂)ₙOH; and polyoxyethylene glyceryl diesters having the formula R₁COOCH₂CH(OOCR₂)CH₂(OCH₂CH₂)ₙOH, wherein R₁ and R₂ are identical or different alkyl, alkenyl, or aromatic hydrocarbon radical which may be substituted or unsubstituted and have between about 4-50 carbon atoms, preferably between about 12-20 carbon atoms, and wherein n is between about 2-80.

Examples of wash-off agents may include: polyoxyethylene (20-80) sorbitan monolaurate, ceteth-2 through ceteth-30, steareth-2 through steareth-30, ceteareth-2 through ceteareth-30, PEG-2 stearate through PEG-30 stearate, PEG-8 distearate, PEG-12 isostearate, C₂₀₋₄₀ pareth-10, C₂₀₋₄₀ pareth-40, PEG-16 hydrogenated castor oil, PEG-40 hydrogenated castor oil, and PEG-20 glyceryl stearate, and mixtures thereof. Examples of commercially available polyoxyethylene (20-80) sorbitan monolaurate are Tween 20 (Polysorbate-20) to Tween 80 (Polysorbate-80) from Sigma-Aldrich, Inc. (St. Louis, Mo.).

The cosmetic or pharmaceutical compositions may include pigments used to provide relatively uniform color to the final cosmetic composition. Pigments may be synthetic or natural. Natural pigments may include pigments or plant-derived colors. Natural pigments may be inorganic (mineral) or organic, white or non-white, and coated or uncoated particles. Natural pigments may include, for example, cerium oxide, chromium oxide, iron oxide, titanium dioxide, zinc oxide, zirconium oxide, carbon black, chromium, chromium hydroxide green, ferric blue, manganese violet, ultramarine blue, D&C and FD&C colors, azo, indigoid, insoluble metallic salts of certified color additives, referred to as the Lakes, and the like, and mixtures thereof.

Compositions herein may include one or more essential and natural oils. Essential oils may be synthetic or natural. Natural essential oils may include bergamot, chamomile german, chamomile maroc, chamomile roman, cinnamon zeylanicum, clove buds, eucalyptus globulus, frankincense, fennel, hyssop, juniper, lemon grass, mountain savory, niaouli, red thyme, rosemary, rose geranium, tagestes, and ylang ylang. Natural oils may include, for example, jojoba oil, sweet almond oil, coconut oil, shea butter, mango butter, and/or aloe vera butter or mixtures thereof.

Synthetic essential oils may include, for example, esters, such as acetylated castor oil, glyceryl stearate, glyceryl dioleate, glyceryl distearate, glyceryl trioctanoate, glyceryl distearate, glyceryl linoleate, glyceryl myristate, glyceryl isostearate, PEG castor oils, PEG glyceryl oleates, PEG glyceryl stearates, PEG glyceryl tallowates, PEG-4 diheptanoate, hydrogenated castor oil, isotridecyl isononanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, tridecyl octanoate, octyl palmitate, dioctyl malate, tridecyl octanoate, myristyl myristate, octododecanol; fatty alcohols such as oleyl alcohol, isocetyl alcohol; and also silicone oils, isoparaffins, hydrogenated polyisobutene, petrolatum, lanolin derivatives, and sorbitan derivatives. Other natural and synthetic oils may be found in the Cosmetic Handbook and CTFA ingredient information.

Compositions herein may include preservatives. Preservatives may be either synthetic or natural and may be used to inhibit growth of undesirable microorganisms. Natural preservatives may include black currant fruit extract, aspen bark, radish root, and sorbic acid, alone or in combination.

Synthetic preservatives may include, for example, methylparaben, ethylparaben, propylparaben, imidazolidinyl urea, diazolidinyl urea, DMDM hydantoin, isothiazolinones, chlorinated aromatic compounds, para-hydroxybenzoic acids/parabens, alone or in combination. Other natural and synthetic preservatives may be found in the Cosmetic Handbook and CTFA ingredient information.

Compositions herein may include gums and powders and mixtures thereof. Gums may include acacia, xanthan, schelortium (amigel), and/or cellulose and mixtures thereof. Powders may include clay, diatomaceous earth, fuller's earth, silica, silica shells or mica, titanated mica, talc, cellulose or spherical cellulose beads, microcrystalline cellulose, corn starch, rice starch, glyceryl starch, soy flour, walnut shell powder, agar, sericite, dextran, nylon, silk powder, chalk, calcium carbonate, bismuth oxychloride, iron oxide, titanium dioxide, aluminum silicate, magnesim aluminum silicate, calcium silicate, aluminum starch, octenylsuccinate, bentonite, hectorite, kaolin, maltodextrin, montmorillonite, zinc laurate, zinc myristate, zinc rosinate, alumina, attapulgite, tin oxide, titanium hydroxide, trimagnesium phosphate of mixtures thereof.

Additional Synthetic ingredients may be found in the compositions herein and include for example, AMP isostearoyl hydrolyzed collagen, AMP isostearoyl hydrolyzed wheat protein, cetyl hydroxyethylcellulose, chondroitin sulfate, cocamidopropyldimethylamine C₈₋₁₆ isoalkysuccinyl lactoglobulin sulfonate, cocodimonium hydroxypropyl hydrolyzed collagen, distarch phosphate, ethyl ester of hydrolyzed animal protein, guar hydroxypropyltrimonium chloride, hydrolyzed animal or plant protein, hydroxypropyl guar, isostearoyl hydrolyzed collagen, and mixtures thereof.

A variety of fragrances and masking agents can be used in these compositions if scented products are desired. Fragrances can be used in an amount in the range of 0-5%, particularly 0.01-2.0%.Masking agents can be used in an amount of 0.05-5.0% (particularly 0.05-2%) by weight based on the total weight of the composition if an unscented product is desired. For reducing whitening in solid compositions liquid or solid high refractive index materials may be used such as diethylhexyl 2,6-naphthalate (from C. P. Hall Co., Chicago, Ill.) or phenyltrimethicone (from Dow Corning Corp., Midland, Mich.) as well as other suitable ingredients.

The invention will be further described in connection with the following illustrative examples.

### Example 1

As shown in Tables 1 and 2, antiperspirant/deodorant active components impregnated in silica powder form a stable stick in combination with a wax structurant and an oil phase.

Antiperspirant/deodorant solutions containing active components were first impregnated in silica powder according to the ratio represented in Table 1 to make silica powder with active ingredients, which is accomplished by mixing the two ingredients of Table 1 in an Osterizer, 12 speed blender.

TABLE 1

| CTFA Name | % (w/w) |
|---|---|
| Al/Zr tetrachlorohydrex. Gly | 97.00 |
| ZnCl₂ Solution | |
| Silica Silyate | 3.00 |
| Total | 100.00 |

Ingredients of the antiperspirant/deodorant sticks were prepared according to Table 2.

TABLE 2

| Seq. | | A | B |
|---|---|---|---|
| No. | CTFA Name | % (w/w) | % (w/w) |
| 1 | Dimethicone (100 cst) | 3.00 | 3.00 |
| 1 | Cyclpentasiloxane (DC 245) | 17.50 | 16.50 |
| 1 | Phenyl trimethicone | 3.00 | 3.00 |
| 1 | C₁₂₋₁₅ alkyl benzoate | 4.00 | 4.00 |
| 2 | Silica powder with actives | 43.00 | 43.00 |
| 3 | Stearyl alcohol | 13.50 | 13.50 |
| 3 | Hydrogenated Castor oil | 5.00 | 6.00 |
| 4 | Silica powder with actives | 10.00 | 10.00 |
| 5 | Fragrance * | 1.00 | 1.00 |
| | Total | 100.00 | 100.00 |

* Magnetic 8110593 mod 4.

The oil phase ingredients represented in Sequence No. 1 were mixed in a beaker. 40% (w/w) of the silica powder with active ingredients was mixed with the oil phase in installments. The resulting mixture was heated to 60° C.

The wax structurants represented in Sequence No. 3 were heated to 80° C. and dissolved, and then cooled to 75° C. The oil phase and silica powder with actives mixture, which was maintained at 60 65° C., was then mixed into the dissolved structurants. The temperature of this mixture dropped to about 62° C.

The mixture was re-heated to 65° C., and then the remaining 10% (w/w) silica powder with actives was mixed-in installments.

Finally, the fragrance represented in Sequence No. 5 was added and mixed; the final mixture was heated to 68° C., and then poured into molds.

An alternate processing method from the one described above is adding wax phase (sequence #3) to the mixture of oil phase and silica powder with actives (sequence #1 and #2)

Smooth and stable antiperspirant/deodorant sticks were formed using ingredients according to A and B and the above-described process.

Antiperspirant/deodorant active solutions (an electrolyte solution of Aluminum Zirconium tetrachlorohydrex. Glycine, Zinc chloride) were impregnated in silica powder according Table 1 to make silica powder with actives. Ingredients of the antiperspirant/deodorant sticks were prepared according to Table 3, then mixed and poured according to the methods used in Example 1, except silica powder with actives were added according to the indicated Sequence Nos.

TABLE 3

| Seq. | | A | B | C | D |
|---|---|---|---|---|---|
| No. | CTFA Name | % (w/w) | % (w/w) | % (w/w) | % (w/w) |
| 1 | Dimethicone (100 cst) | 4.00 | 5.00 | 4.00 | 4.00 |
| 1 | Cyclpentasiloxane | 15.50 | 20.00 | 17.50 | 17.50 |
| | (DC 245) | | | | |
| 1 | Phenyl trimethicone | 3.00 | 3.00 | 3.00 | 3.00 |
| 1 | C₁₂₋₁₅ alkyl benzoate | 4.00 | 4.00 | 4.00 | 4.00 |
| 2 | Silica powder with actives | 53.00 | 53.00 | 53.00 | 0.00 |
| 3 | Stearyl alcohol | 13.00 | 13.00 | 13.00 | 13.00 |
| 3 | Hydrogenated Castor oil | 6.00 | 5.00 | 4.50 | 4.50 |
| 4 | Silica Silyate | 0.00 | 0.00 | 0.00 | 1.59 |
| | Al/Zr tetrachlorohydrex. | 0.00 | 0.00 | 0.00 | 51.41 |
| 4 | Gly ZnCl₂ Solution | | | | |
| 5 | Fragrance * | 1.00 | 1.00 | 1.00 | 1.00 |
| | Total | 100.00 | 100.00 | 100.00 | 100.00 |

* Magnetic 8110593 mod 4.

Smooth and stable antiperspirant/deodorant sticks were formed using ingredients according to A, B, and C by the above-described process. In the case of D no stick was formed, the aqueous solution of Al/Zr tetrachlorohydrex, Gly ZnCl2 separated.

Antiperspirant/deodorant active solutions were impregnated in silica powder according Table 1 to make silica powder with actives. Ingredients of the antiperspirant/deodorant sticks were prepared according to Table 4, then mixed and poured according to the methods used in Example 1, except silica powder with actives were added according to the indicated Sequence No.

TABLE 4

| Seq. | | A | B |
|---|---|---|---|
| No. | CTFA Name | % (w/w) | % (w/w) |
| 1 | Dimethicone (100 cst) | 4.00 | 4.00 |
| 1 | Cyclpentasiloxane (DC 245) | 16.50 | 15.50 |
| 1 | Phenyl trimethicone | 3.00 | 3.00 |
| 1 | C₁₂₋₁₅ alkyl benzoate | 4.00 | 4.00 |
| 2 | Silica powder with actives | 53.00 | 53.00 |
| 3 | Stearyl alcohol | 11.00 | 11.00 |
| 3 | AMS-C30 Cosmetic wax | 2.50 | 0.00 |
| 3 | Synthetic wax (C₂₀₋₄₀) | 0.00 | 2.50 |
| 3 | Hydrogenated Castor oil | 5.00 | 6.00 |
| 4 | Fragrance * | 1.00 | 1.00 |
| | Total | 100.00 | 100.00 |

* Magnetic 8110593 mod 4.

Smooth and stable antiperspirant/deodorant sticks were formed using ingredients according to A and B and the above-described process.

Antiperspirant/deodorant active solutions were impregnated in silica powder according Table 1 to make silica powder with actives. Ingredients of the antiperspirant/deodorant sticks were prepared according to Table 5, then mixed and poured according to the methods used in Example 1, except silica powder with actives were added according to the indicated Sequence No.

TABLE 5

| Seq. | | A | B |
|---|---|---|---|
| No. | CTFA Name | % (w/w) | % (w/w) |
| 1 | Dimethicone (100 cst) | 4.00 | 4.00 |
| 1 | Cyclpentasiloxane (DC 245) | 16.50 | 15.50 |
| 1 | Phenyl trimethicone | 3.00 | 3.00 |
| 1 | C₁₂₋₁₅ alkyl benzoate | 4.00 | 4.00 |
| 2 | Silica powder with actives | 53.00 | 53.00 |
| 3 | Stearyl alcohol | 11.00 | 11.00 |
| 3 | Synthetic wax (C₂₀₋₄₀) | 1.50 | 2.50 |
| 3 | Hydroxy stearic acid “B” | 6.00 | 6.00 |
| 4 | Fragrance * | 1.00 | 1.00 |
| | Total | 100.00 | 100.00 |

* Magnetic 8110593 mod 4.

Smooth, stable, but softer antiperspirant/deodorant sticks were formed using ingredients according to A and B and the above-described process.

Antiperspirant/deodorant active solutions were impregnated in silica powder according Table 1 to make silica powder with actives. Ingredients of the antiperspirant/deodorant sticks were prepared according to Table 6, then mixed and poured according to the methods used in Example 1, except silica powder with actives were added according to the indicated Sequence No.

TABLE 6

| Seq. | | A | B |
|---|---|---|---|
| No. | CTFA Name | % (w/w) | % (w/w) |
| 1 | Dimethicone (100 cst) | 4.00 | 4.00 |
| 1 | Cyclpentasiloxane (DC 245) | 16.50 | 15.50 |
| 1 | Phenyl trimethicone | 3.00 | 3.00 |
| 1 | C₁₂₋₁₅ alkyl benzoate | 4.00 | 4.00 |
| 2 | Silica powder with actives | 53.00 | 53.00 |
| 3 | Stearyl alcohol | 11.00 | 11.00 |
| 3 | Synthetic wax (C₂₀₋₄₀) | 1.50 | 2.50 |
| 3 | Hydrogenated Castor oil | 5.50 | 5.00 |
| 3 | Petrolatum | 0.50 | 1.00 |
| 5 | Fragrance * | 1.00 | 1.00 |
| | Total | 100.00 | 100.00 |

* Magnetic 8110593 mod 4.

Smooth and stable antiperspirant/deodorant sticks were formed using ingredients according to A and B and the above-described process.

TABLE 7

| CTFA Name | % (w/w) |
|---|---|
| Al/Zr tetrachiorohydrex. Gly | 97.00 |
| Solution | |
| Silica Silyate | 3.00 |
| Total | 100.00 |

Antiperspirant/deodorant active solutions were impregnated in silica powder according Table 7 to make silica powder with actives. Ingredients of the antiperspirant/deodorant sticks were prepared according to Table 8, then mixed and poured according to the methods used in Example 1, except silica powder with actives were added according to the indicated Sequence No.

TABLE 8

| Seq. | | A | B |
|---|---|---|---|
| No. | CTFA Name | % (w/w) | % (w/w) |
| 1 | Dimethicone (100 cst) | 5.00 | 5.00 |
| 1 | Cyclpentasiloxane (DC-245) | 0.00 | 7.80 |
| 1 | DC-2-1184 fluid | 8.00 | 0.00 |
| 1 | C₁₂₋₁₅ alkyl benzoate | 12.00 | 12.00 |
| 2 | Silica powder with actives | 53.00 | 53.00 |
| 3 | Stearyl alcohol | 13.00 | 13.00 |
| 3 | Hydrogenated Castor oil | 7.00 | 7.00 |
| 3 | Polysorbate-20 | 0.50 | 0.50 |
| 3 | Dibutyl Lauroyl Glutamide | 0.20 | 0.00 |
| 3 | Dibutyl Ethylhexanoyl | 0.30 | 0.70 |
| | Glutamide | | |
| 4 | Fragrance * | 1.00 | 1.00 |
| | Total | 100.00 | 100.00 |

* Magnetic 8110593 mod 4.

Smooth and stable antiperspirant/deodorant sticks were formed using ingredients according to A and B and the above-described process.

Antiperspirant/deodorant active solutions were impregnated in silica powder according Table 7 to make silica powder with actives. Ingredients of the antiperspirant/deodorant sticks were prepared according to Table 9, then mixed and poured according to the methods used in Example 1, except silica powder with actives were added according to the indicated Sequence No.

TABLE 9

| Seq. | | A |
|---|---|---|
| No. | CTFA Name | % (w/w) |
| 1 | Dimethicone (100 ct) | 5.00 |
| 1 | DC-2-1184 fluid | 8.00 |
| 1 | DC-5562 (carbinol fluid) | 6.00 |
| 1 | C₁₂₋₁₅ alkyl benzoate | 5.50 |
| 1 | Polysorbate-20 | 0.50 |
| 2 | Silica powder with actives | 53.00 |
| 3 | Stearyl alcohol | 14.00 |
| 3 | Hydrogenated Castor oil | 7.00 |
| 4 | Fragrance * | 1.00 |
| | Total | 100.00 |

* Magnetic 8110593 mod 4.

Smooth and stable antiperspirant/deodorant sticks were formed using ingredients according to A and the above-described process that have low residue and good application aesthetics.

Antiperspirant/deodorant active solutions were impregnated in silica powder according Table 7 to make silica powder with actives. Ingredients of the antiperspirant/deodorant sticks were prepared according to Table 10, then mixed and poured according to the methods used in Example 1, except silica powder with actives were added according to the indicated Sequence No.

TABLE 10

| Seq. | | | A |
|---|---|---|---|
| No. | CTFA Name | B | % (w/w) |
| 1 | Dimethicone (100 cst) | 5.00 | 5.00 |
| 1 | Hydrogenated | 1.00 | 1.00 |
| | polyisobutene | | |
| 1 | Ethyl Macadamiate | 0.00 | 7.50 |
| | (Floramac-10) | | |
| 1 | Cyclopentasiloxane | 7.50 | 0 |
| | (DC 245) | | |
| 1 | C₁₂₋₁₅ alkyl benzoate | 11.00 | 11.0 |
| 2 | Silica powder with actives | 53.00 | 53.00 |
| 3 | Stearyl alcohol | 14.00 | 14.00 |
| 3 | Hydrogenated Castor oil | 7.00 | 7.00 |
| 1 | Polysorbate-20 | 5.00 | 0.50 |
| 4 | Fragrance * | 1.00 | 1.00 |
| | Total | 100.00 | 100.00 |

* Magnetic 8110593 mod 4.

Smooth and stable antiperspirant/deodorant sticks were formed using ingredients according to A and the above described process. In column A Floramac-10 (Ethyl Macadamiate, Tocopherol, and Malic Acid) replaces cyclopentasiloxane found in the formula shown in column B Table 10.

Stable antiperspirant/deodorant sticks were also formed when using ingredients according to A, B and C in TABLE 11 and the above-described process. In these instances DC-9041 and DC-9040, two, non-emulsifying elastomers are incorporated in the formula.

TABLE 11

| | | A | B | C |
|---|---|---|---|---|
| Seq # | CTFA Name | % | % | % |
| 1 | Dimethicone (100 cst) | 4.00 | 4.00 | 4.00 |
| 1 | DC-2-1184 fluid | 7.00 | 0.00 | 0.00 |
| 1 | Cyclopentasiloxane (DC 245) | 0.00 | 7.00 | 7.00 |
| 1 | Dermol | 7.00 | 7.00 | 7.00 |
| 1 | Phenyl trimethicone | 3.00 | 3.00 | 3.00 |
| 1 | C12-15 alkyl benzoate | 4.00 | 4.00 | 4.00 |
| 1 | DC-9041 | 3.00 | 3.00 | 0.00 |
| 1 | DC-9040 | 0.00 | 0.00 | 3.00 |
| 2 | Silica powder/w above 97:3 (Table 7) | 53.00 | 53.00 | 53.00 |
| 3 | stearyl alcohol | 13.00 | 13.00 | 13.00 |
| 3 | Hydrogenated Caster wax | 5.00 | 5.00 | 5.00 |
| 4 | Fragrance | 1.00 | 1.00 | 1.00 |

Examples of sticks containing polyethylene (Performalene 400 PE) and cosmetic wax as well as alternate a long chain esters such as isononyl isononaoate and an alkyl substituted dimethicone such as caprylyl methicone are shown in examples A, B and C of Table 12 The structuring aids further optimize payout and stick hardness. To process sticks that contain these higher melting structurants, the wax phase (represented by ingredients listed as sequence #3) should be heated to 80 to 95 C and cooled down to 80 to 85 C before mixing with the oil phase containing the silica powder with active ingredients (sequence #2) Additionally, the temperature of the mixture of the oil phase and silica powder with actives (sequence #1 and 2) should be such that the wax phase (sequence #3) does not solidify or precipitate out. Typically this temperature ranges from 62 to 80 C.

TABLE 12

| | | A | B | |
|---|---|---|---|---|
| | CTFA Name | % | % | C |
| 1 | Dimethicone (100 cst) | 5.80 | 5.80 | 5.80 |
| 1 | Dimethicone 5 cst | 0.00 | 0.00 | 5.00 |
| 1 | DC 246 | 5.00 | 5.00 | 0.00 |
| 1 | Caprylyl Methicone | 2.50 | 0.00 | 2.50 |
| 1 | C12-15 alkyl benzoate | 9.30 | 9.30 | 9.30 |
| 1 | Isononyl lsononaoate | 0.00 | 2.50 | 0.00 |
| 1 | Polysorbate-20 | 0.50 | 0.50 | 0.50 |
| 2 | Silica powder/w above 97:3 | 53.00 | 53.00 | 53.00 |
| 3 | stearyl alcohol | 14.00 | 14.00 | 14.00 |
| 3 | Hydrogenated Castor wax | 7.00 | 7.00 | 7.00 |
| 3 | AMS-C30 Cosmetic Wax | 0.50 | 0.50 | 0.50 |
| 3 | Performalene 400PE | 1.00 | 1.00 | 1.00 |
| | Fragrance | 1.40 | 1.40 | 1.40 |
| | Total | 100.00 | 100.00 | |

Antiperspirant/deodorant active solutions were first impregnated in silica powder according to the ratio represented in Table 13 to make silica powder with actives. The resulting silica powders with actives clumped together

TABLE 13

| CTFA Name | % (w/w) |
|---|---|
| Al/Zr tetrachlorohydrex. Gly | 99.30 |
| Solution | |
| Silica Silyate | 0.70 |
| Total | 100.00 |

Ingredients of the antiperspirant/deodorant sticks were prepared according to Table 14, using the silica powder with actives prepared in Table 13. The formula was mixed and poured according to the methods used in Example 1, except silica powder with actives (from Table 10) were added according to the indicated Sequence No.

TABLE 14

| Seq. | | A |
|---|---|---|
| No. | CTFA Name | % (w/w) |
| 1 | Dimethicone (100 cst) | 5.00 |
| 1 | Cyclpentasiloxane (DC 245) | 9.00 |
| 1 | Dermal 89 | 6.00 |
| 1 | Phenyl trimethicone | 3.00 |
| 1 | C₁₂₋₁₅ alkyl benzoate | 6.00 |
| 2 | Silica powder with actives | 53.00 |
| 3 | Stearyl alcohol | 13.00 |
| 3 | Hydrogenated Castor oil | 7.00 |
| 4 | Fragrance * | 1.00 |
| | Total | 100.00 |

* Magnetic 8110593 mod 4.

No antiperspirant/deodorant sticks were formed. The aqueous phase started to separate from the oil phase. This example shows that the soluble cosmetic actives to retentive filler ratio should be below about 142:1, and preferably below about 99:1, in order form a stable stable silica powder with actives and consequently a stable solid cosmetic composition.

Antiperspirant/deodorant active solutions were impregnated in silica powder according Table 1 to make silica powder with actives. Ingredients of the antiperspirant/deodorant sticks were prepared according to Table 15, then mixed and poured according to the methods used in Example 1, except silica powder with actives were added according to the indicated Sequence No,

TABLE 15

| Seq. | | A |
|---|---|---|
| No. | CTFA Name | % (w/w) |
| 1 | Dermol 89 | 1.00 |
| 1 | Cyclpentasiloxane (DC 245) | 9.00 |
| 1 | Dipropylene glycol | 10.00 |
| 1 | C₁₂₋₁₅ alkyl benzoate | 7.00 |
| 2 | Silica powder with actives | 53.00 |
| 3 | Stearyl alcohol | 13.00 |
| 3 | Hydrogenated Castor oil | 6.00 |
| 4 | Fragrance * | 1.00 |
| | Total | 100.00 |

No antiperspirant/deodorant sticks were formed; the aqueous phase and the oil phase separated into two layers. This example shows that the presence of dipropylene glycol disrupts the formation of the solid cosmetic composition.

Antiperspirant/deodorant active solutions were first impregnated in silica powder according to the ratio represented in Table 16 to make silica powder with actives.

TABLE 16

| | A | B | C |
|---|---|---|---|
| CTFA Name | % (w/w) | % (w/w) | % (w/w) |
| Al/Zr tetrachlorohydrex. Gly | 97.00 | 0.00 | 0.00 |
| Solution | | | |
| Water (H₂O) | 0.00 | 97.00 | 94.00 |
| Silica Silyate | 3.00 | 3.00 | 6.00 |
| Total | 100.00 | 100.00 | 100.00 |

Mixtures according to A or C created free flowing silica powder with impregnated liquids. No free flowing powder was created using the mixture according to B.

Ingredients of the antiperspirant/deodorant sticks were prepared according to Table 17, The silica powders of Columns A, B, and C in Table 16 were used for the corresponding Column letters in Table 17. The ingredients were mixed and poured according to the methods used in Example 1, except the silica powders with actives were added according to the indicated Sequence No.

TABLE 17

| Seq. | | A | B | C |
|---|---|---|---|---|
| No. | CTFA Name | % (w/w) | % (w/w) | % (w/w) |
| 1 | Dimethicone (100 cst) | 5.00 | 5.00 | 5.00 |
| 1 | DC-2-1184 fluid | 8.00 | 8.00 | 6.00 |
| 1 | C₁₂₋₁₅ alkyl benzoate | 11.50 | 11.50 | 6.50 |
| 1 | Polysorbate-20 | 0.50 | 0.50 | 0.50 |
| 2 | Silica powder with liquid | 53.00 | 53.00 | 60.00 |
| 3 | Stearyl alcohol | 14.00 | 14.00 | 14.00 |
| 3 | Hydrogenated Castor oil | 7.00 | 7.00 | 7.00 |
| 4 | Fragrance * | 1.00 | 1.00 | 1.00 |
| | Total | 100.00 | 100.00 | 100.00 |

* Magnetic 8110593 mod 4.

Normal smooth and stable antiperspirant/deodorant sticks were formed according to A. No sticks were formed according to B or C. The aqueous phase started to separate from the oil phase in the mixture according to C. This example shows that in the absence of electrolytes, the intended solid cosmetic composition cannot be formed.

Antiperspirant/deodorant active solutions were impregnated in silica powder according Table 7 to make silica powder with actives. Ingredients of the antiperspirant/deodorant sticks were prepared according to Table 18, then mixed and poured according to the methods used in Example 1, except silica powder with actives were added according to the indicated Sequence No.

TABLE 18

| Seq. | | A | B | C |
|---|---|---|---|---|
| No. | CTFA Name | % (w/w) | % (w/w) | % (w/w) |
| 1 | Dimethicone 100 cst | 0.00 | 5.00 | 5.00 |
| 1 | Dimethicone 5 cst | 0.00 | 0.00 | 7.30 |
| 1 | Hydrogenated polyisobutene | 0.00 | 1.00 | 1.00 |
| 1 | C₁₂₋₁₅ alkyl benzoate | 17.40 | 9.00 | 9.00 |
| 1 | DC-246 | 6.70 | 7.10 | 0.00 |
| 1 | Polysorbate-20 | 0.50 | 0.50 | 0.50 |
| 2 | Silica powder with actives | 53.00 | 53.00 | 53.00 |
| 3 | AMS-C30 Cosmetic Wax | 0.00 | 1.50 | 1.00 |
| 3 | Stearyl alcohol | 14.00 | 14.00 | 14.00 |
| 3 | Hydrogenated Castor oil | 0.00 | 7.00 | 7.00 |
| 3 | Performacol 425 alcohol | 7.00 | 0.50 | 1.00 |
| 4 | Fragrance * | 1.40 | 1.40 | 1.20 |
| | Total | 100.00 | 100.00 | 100.00 |

* Magnetic 8110593 mod 4.

Smooth and stable antiperspirant/deodorant sticks were formed using ingredients according to A, B and C and the above-described process, which used a long carbon chain linear alcohol (Performacol 425 alcohol from New Phase Technologies) as a co-structurant to adjust the stick hardness.

The aluminum zirconium salt solution was impregnated in silica powder according Table 7 to make silica powder with the electrolytes. Ingredients of the stick containing an oil soluble active was prepared according to Table 19, then mixed and poured according to the methods used in Example 1, except silica powder with actives were added according to the indicated Sequence No.

TABLE 19

| Seq. | | A |
|---|---|---|
| No. | CTFA Name | % (w/w) |
| 1 | Dimethicone 100 cst | 5.80 |
| | Dimethicone 5 cst | 0.00 |
| 1 | Octyl methoxy-cinnamate | 3.00 |
| 1 | C₁₂₋₁₅ alkyl benzoate | 17.40 |
| 1 | DC-246 | 11.40 |
| 1 | Polysorbate-20 | 0.50 |
| 2 | Silica powder with actives | 45.00 |
| 3 | AMS-C30 Cosmetic Wax | 0.50 |
| 3 | Stearyl alcohol | 14.00 |
| 3 | Hydrogenated Castor oil | 7.00 |
| 3 | Performalene 400 PE | 1.00 |
| 3 | Performacol 425 alcohol | 7.00 |
| 4 | Fragrance * | 1.40 |
| | Total | 100.00 |

* Magnetic 8110593 mod 4. Internal ref: Bk# 822-010A

A smooth and stable stick containing an oil soluble cosmetic active was prepared. For illustrative purposes the sunscreen agent octylmethoxy cinnamate was incorporated using ingredients according to A, and the above-described process.

What is claimed is:

1. A cosmetic or pharmaceutical composition comprising:
one or more cosmetic or pharmaceutical actives in a concentration of about 1% to 60% of the total formula wherein said cosmetic or pharmaceutical active is;
solubilized in an electrolyte solution free of water-absorbing polymers, dipropylene glycol, and emulsifiers and is
impregnated within one or more retentive fillers,
has one or more structurants having a melting point higher than about 50° C.,
and an oil phase selected from the group consisting of organic or silicone compounds or a combination of both such that the oil phase is greater than about 10% w/w and less than about 80% w/w, and
said composition is capable of forming a solid composition.

2. The composition of claim 1, wherein the water soluble cosmetic or pharmaceutical active is in a concentration of 15 to 45%.

3. The composition of claim 1, wherein the water soluble cosmetic or pharmaceutical active to retentive filler ratio is less than about 142:1.

4. The composition of claim 1, wherein the water soluble cosmetic or pharmaceutical active to retentive filler ratio is between about 7:1 and 99:1.

5. The composition of claim 1, wherein the retentive filler is selected from the group comprising silica, hydrophobically modified silica, lamellar filler, crosspolymer, cellulosic, resins, silicone elastomers and any combination thereof.

6. The composition of claim 1, wherein the one or more structurants are in a concentration of 0.1-50%.

7. The composition of claim 1, wherein the oil phase comprises volatile oils, non-volatile oils, or a combination of both.

8. The composition of claim 1, wherein the composition optionally includes excipients, pigments, essential oils, natural oils, wash-off agents, preservatives, gums, powders, synthetic ingredients, fragrances, masking agents, high refractive index material, sunscreen and combinations thereof.

9. The composition of claim 1, wherein the one or more structurant is selected from the group consisting of waxes, fiber-forming gellants, silicone waxes and combinations thereof.

10. The Process of preparing the composition of claim 1 comprising:
a. add active and silica powder;
b. optionally mix and add a portion of (a) to the oil phase;
c. add (b) to the structurant and optionally cool;
d. reheat (c) and add the remaining (a);
e. optionally add additional ingredients; and
f. heat (e) until it forms a pourable liquid and pour in molds to obtain a solid composition.

11. The Process of claim 10, wherein step (c) is merged with step (b).

12. The Process of claim 10 wherein step (b) is merged with step (a) and step (d) comprises only reheating.
